(19) United States
(12) Patent Application Publication
(10) Pub. No.: US 2018/0338695 A1
(43) Pub. Date: Nov. 29, 2018

(54) SYSTOLIC PRESSURE CALIBRATION

(71) Applicant: Edwards Lifesciences Corporation, Irvine, CA (US)

(72) Inventors: Berend Westerhof, Amstelveen (NL); Olaf Schraa, Amsterdam (NL); Christianus Joannes Jozef Eugène van Groeningen, Utrecht (NL); Peiyuan Li, Amsterdam (NL); Jeroen Van Goudoever, Amstelveen (NL)

(21) Appl. No.: 15/984,684

(22) Filed: May 21, 2018

Related U.S. Application Data
(60) Provisional application No. 62/510,351, filed on May 24, 2017.

(51) Int. Cl. A61B 5/0225; A61B 5/022

(52) U.S. Cl. CPC A61B 5/02255; A61B 2560/0223; A61B 5/02241

(57) ABSTRACT

Disclosed is a method for measuring systolic arterial blood pressure of a patient utilizing a finger cuff including an inflatable bladder and a pulsatility sensor that detects arterial pulsatility in a finger of the patient, the method comprising: placing the finger cuff around the finger of the patient; inflating the bladder of the finger cuff until the finger cuff applies a first pressure to the finger, the first pressure being greater than the systolic pressure to be measured; deflating the bladder of the finger cuff until the finger cuff applies a second pressure to the finger, the second pressure being less than the systolic pressure to be measured; monitoring the arterial pulsatility in the finger with the pulsatility sensor; and obtaining a measurement of the systolic arterial blood pressure based on pressure applied to the finger by the finger cuff and the arterial pulsatility in the finger.

### CROSS-REFERENCE TO RELATED APPLICATIONS

This application claims priority to U.S. Provisional Patent Application No. 62/510,351, filed May 24, 2017, the entire contents of which are incorporated herein by reference.

### BACKGROUND

### Field

Embodiments of the invention relate to non-invasive blood pressure measurement using a finger cuff and a volume clamp method and that utilizes systolic pressure calibration.

### Relevant Background

Volume clamping is a technique for non-invasively measuring blood pressure in which pressure is applied to a subject's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure.

In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in conjunction with the measurement of the arterial volume. To accurately measure blood pressure, the feedback loop provides sufficient pressure generating and releasing capabilities to match the pressure oscillations of the subject's blood pressure.

Calibrating a volume clamp system with known methods may be difficult and/or cumbersome. In the prior art, an algorithm has been described that switches between volume clamp blood pressure measurement and periods with a constant cuff pressure, during which the shape of plethysmographic waveform is analyzed to determine the plethysmogram corresponding to an unloaded artery (EP 0080778 B1). The unloaded volume state can also found by introducing a high frequency disturbance on the pressure signal during volume clamp mode (EP 0284095 B1). By analyzing the corresponding plethysmographic changes the volume with maximal dynamic ‘Gefäβkomplianz’ compliance can be located. Another method to calibrate a volume clamp blood pressure measurement is to first find an approximation of the unloaded volume (e.g., by determining at which cuff pressure level the beat-to-beat amplitude of the plethysmogram is largest) and then to perform a calibration with an independent reference blood pressure (such as an oscillometric blood pressure measurement with upper arm cuff). An upper arm cuff can also be used to determine systolic pressure if the arm cuff is first inflated to a pressure level above systolic pressure, and then is gradually reduced, and by subsequently determining at which cuff pressure the heart pulsations can be first detected in the finger blood pressure signal or plethysmogram (U.S. Pat. No. 5,746,698 A). Due to a gradual pressure decay and pressure waveform distortion towards the periphery the finger blood pressure may deviate from arm cuff pressure, and currently there is no good way to calibrate a finger blood pressure volume clamp measurement with sufficient accuracy.

### SUMMARY

Embodiments of the invention may relate to a method for measuring systolic arterial blood pressure of a patient utilizing a finger cuff including an inflatable bladder and a pulsatility sensor that detects arterial pulsatility in a finger of the patient, the method comprising: placing the finger cuff around the finger of the patient; inflating the bladder of the finger cuff until the finger cuff applies a first pressure to the finger, the first pressure being greater than the systolic pressure to be measured; deflating the bladder of the finger cuff until the finger cuff applies a second pressure to the finger, the second pressure being less than the systolic pressure to be measured; monitoring the arterial pulsatility in the finger with the pulsatility sensor; and obtaining a measurement of the systolic arterial blood pressure based on pressure applied to the finger by the finger cuff and the arterial pulsatility in the finger. As will be described, various techniques are utilized to obtain the measurement of the systolic arterial blood pressure which is used to calibrate the arterial blood pressure measurement system.

### BRIEF DESCRIPTION OF THE DRAWINGS

FIG. 1 is a diagram of an example of a blood pressure measurement system.

FIG. 2 is a block diagram illustrating an example environment in which embodiments of the invention may be practiced.

FIG. 3 is a block diagram illustrating example control circuitry.

FIG. 4 is a flowchart illustrating an example method for measuring a finger systolic arterial blood pressure.

FIG. 5 is a diagram illustrating various measurement traces according to embodiments of the invention.

FIG. 6 is a block diagram illustrating an example environment in which embodiments of the invention may be practiced.

### DETAILED DESCRIPTION

Embodiments of the invention may relate to a method for measuring finger systolic arterial blood pressure, comprising: placing a finger cuff around a finger of a patient, the finger cuff comprising an inflatable bladder and a pulsatility sensor that detects arterial pulsatility in the finger; inflating the bladder of the finger cuff until the finger cuff applies a first pressure to the finger, the first pressure being greater than the systolic pressure to be measured; deflating the bladder of the finger cuff until the finger cuff applies a second pressure to the finger, the second pressure being less than the systolic pressure to be measured; monitoring the arterial pulsatility in the finger with the pulsatility sensor; and obtaining a measurement of the finger systolic arterial blood pressure based on pressure applied to the finger by the finger cuff and the arterial pulsatility in the finger. As will be described, various techniques are utilized to obtain the measurement of the systolic arterial blood pressure which is used to calibrate the arterial blood pressure measurement system.

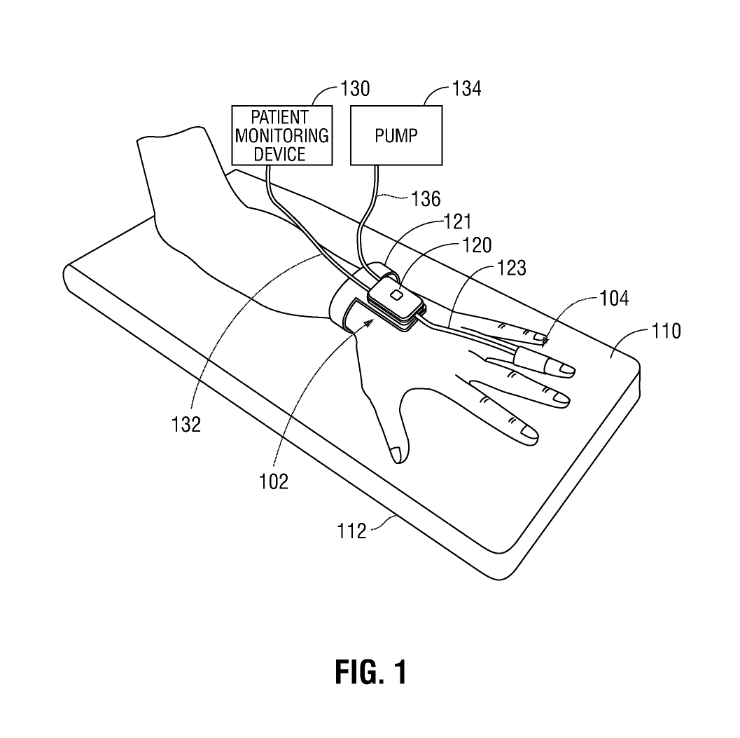

With reference to FIG. 1, an example of an environment in which a finger cuff 104 may be implemented will be described. As an example, a blood pressure measurement system 102 that includes a finger cuff 104 that may be attached to a patient's finger and a blood pressure measurement controller 120 that may be attached to the patient's body (e.g., a patient's wrist or hand) is shown. The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuity. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure senor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130. In some embodiments, a blood pressure measurement controller 120 is not used at all and there is simply a connection from the tube 123 to finger cuff connector 122 from a remote pump 134 including a remote pressure regulatory system, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system that can be used, including but not limited to the blood pressure measurement controller, may be described simply as a pressure generating and regulating system. As a further example, in some embodiments, there may be no blood pressure measurement controller, at all, and a remote pump 134 that is controlled remotely may be directly connected via a tube 136 and 123 to finger cuff 104 to provide pneumatic pressure to the finger cuff 104.

Calibrating a volume clamp method-based non-invasive finger arterial blood pressure measurement system, such as the system illustrated in FIG. 1, may be difficult and/or cumbersome. Systolic pressure has always been the most difficult pressure to obtain precisely from the finger arterial pressure.

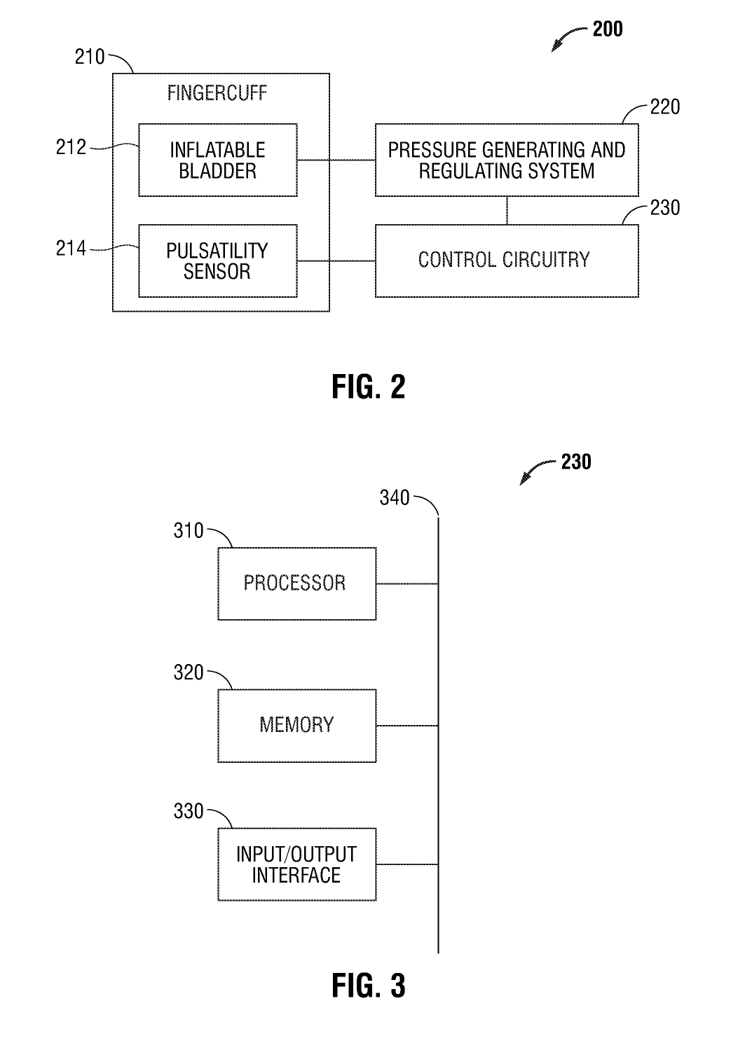

Referring to FIG. 2, a block diagram illustrating an example environment 200 in which embodiments of the invention may be practiced is shown. The finger cuff 210 may comprise an inflatable bladder 212 and an arterial pulsatility sensor 214. The inflatable bladder 212 may be pneumatically connected to a pressure generating and regulating system 220. The pressure generating and regulating system 220 may generate, measure, and regulate pneumatic pressure that inflates or deflates the bladder 212, and may comprise such elements as a pump, a valve, a sensor, control circuitry, and/or other suitable elements. When the bladder 212 is inflated, the finger cuff 210 applies a pressure to the finger. The pressure applied to the finger by the finger cuff 210 may be the same as the pneumatic pressure in the bladder 212.

In one embodiment, the arterial pulsatility sensor 214 may comprise a plethysmograph. The plethysmograph may make continuous volumetric measurements (or plethysmogram) of arterial blood flows within the finger. Thus, pulsatility in the finger may be detected based on the plethysmogram. In one embodiment, the plethysmograph may comprise a light-emitting diode (LED)—photodiode pair. The LED may be used to illuminate the finger skin and light absorption or reflection may be detected with the photodiode. Therefore, the plethysmogram may be generated based on the signal received from the photodiode.

The pressure generating and regulating system 220 and the arterial pulsatility sensor 214 may be connected to a control circuitry 230. The control circuitry 230 may instruct the pressure generating and regulating system 220 to inflate or deflate the bladder 212 based on a pressure setting, may receive pulsatility information from the pulsatility sensor 214, and may carry out necessary data manipulations.

Referring to FIG. 3, a block diagram illustrating example control circuitry 230 is shown. It should be appreciated that FIG. 3 illustrates a non-limiting example of a control circuitry 230 implementation. Other implementations of the control circuitry 230 not shown in FIG. 3 are also possible. The control circuitry 230 may comprise a processor 310, a memory 320, and an input/output interface 330 connected with a bus 340. Under the control of the processor 310, data may be received from an external source through the input/output interface 330 and stored in the memory 320, and/or may be transmitted from the memory 320 to an external destination through the input/output interface 330. The processor 310 may process, add, remove, change, or otherwise manipulate data stored in the memory 320. Further, code may be stored in the memory 320. The code, when executed by the processor 310, may cause the processor 310 to perform operations relating to data manipulation and/or transmission and/or any other possible operations.

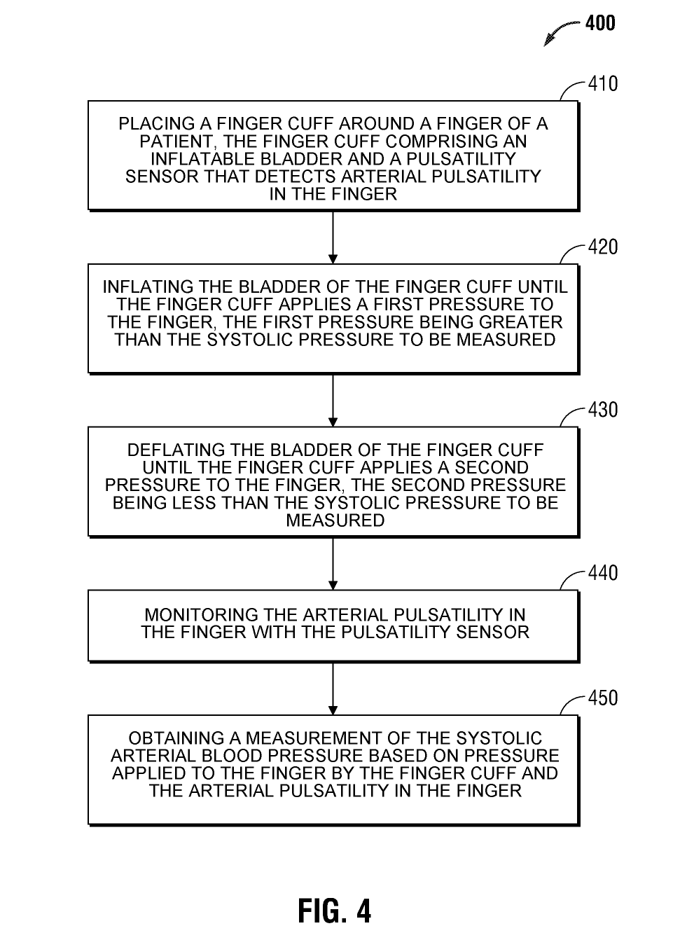

Referring to FIG. 4, a flowchart illustrating an example method 400 for measuring a finger systolic arterial blood pressure is shown. At block 410, a finger cuff may be placed around a finger of a patient. The finger cuff may comprise an inflatable bladder and a pulsatility sensor that detects arterial pulsatility in the finger. At block 420, the bladder of the finger cuff may be inflated until the finger cuff applies a first pressure to the finger, the first pressure being greater than the systolic pressure to be measured. At block 430, the bladder of the finger cuff may be deflated until the finger cuff applies a second pressure to the finger, the second pressure being less than the systolic pressure to be measured. At block 440, the arterial pulsatility in the finger may be monitored with the pulsatility sensor. At block 450, a measurement of the finger systolic arterial blood pressure may be obtained based on pressure applied to the finger by the finger cuff and the arterial pulsatility in the finger. The measured finger systolic arterial blood pressure may be used to calibrate a continuous arterial blood pressure measurement system.

Further, as will be described, embodiments include measuring a pressure (e.g., a third pressure) applied to the finger by the finger cuff at a first time instant when the arterial pulsatility in the finger disappears when the bladder of the finger cuff is inflated to determine the finger systolic arterial blood pressure that may be used to calibrate the continuous arterial blood pressure measurement system. Additionally, embodiments include measuring a pressure (e.g., a fourth pressure) applied to the finger by the finger cuff at a second time instant when the arterial pulsatility in the finger resumes when the bladder of the finger cuff is deflated to determine the finger systolic arterial blood pressure that may be used to calibrate the continuous arterial blood pressure measurement system. Moreover, in one embodiment, an average of the third pressure and the fourth pressure may be used to determine the finger systolic arterial blood pressure that may be used to calibrate the continuous arterial blood pressure measurement system.

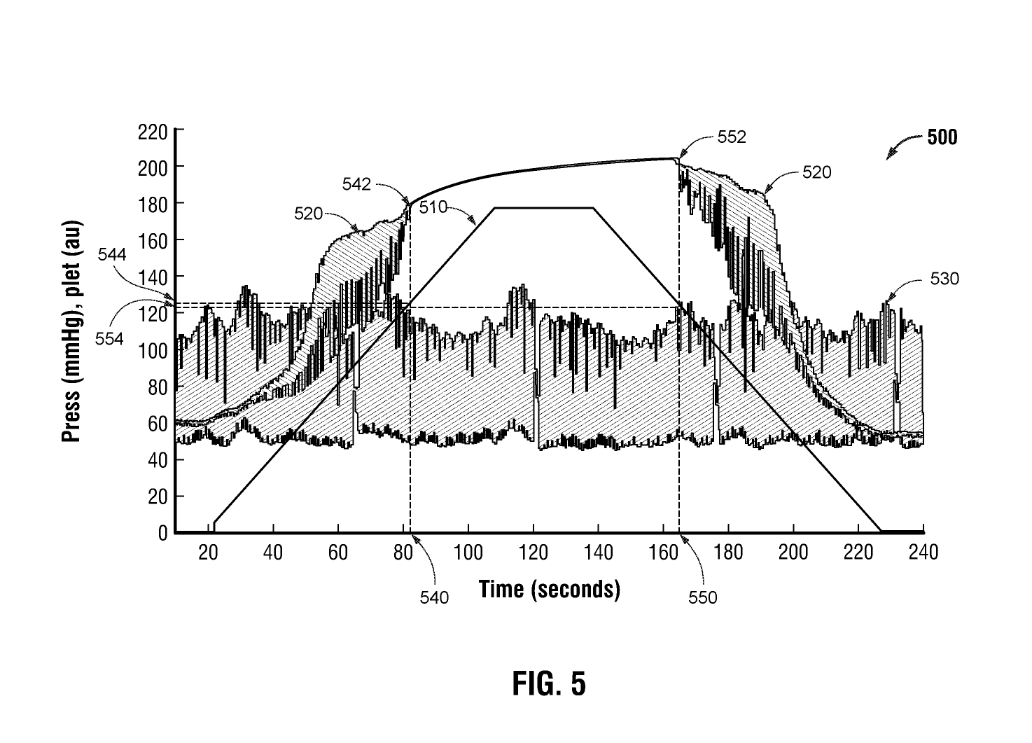

Referring to FIG. 5, a diagram 500 illustrating various measurement traces according to embodiments of the invention is shown. FIG. 5 shows that measurements obtained according to the example method 400 correspond to the systolic arterial pressure. Three measurement traces are shown in FIG. 5: a first trace 510, a second trace 520, and a third trace 530. The first trace 510 shows the pneumatic pressure within the bladder 212, which corresponds to the pressure applied to the finger by the finger cuff 210. The second trace 520 shows the plethysmogram obtained from the pulsatility sensor 214 (e.g., a plethysmograph). Further, the third trace 530 shows the continuous blood pressure measured with the volume clamp method.

As can be seen in FIG. 5, the bladder of the finger cuff may be inflated until the finger cuff applies a first pressure to the finger (trace 510) (e.g., above point 544), the first pressure being greater than the systolic pressure to be measured. Further, the bladder of the finger cuff may be deflated until the finger cuff applies a second pressure to the finger (trace 510) (e.g., below point 554), the second pressure being less than the systolic pressure to be measured.

In particular, when the bladder 212 is inflated (the rising segment of the first trace 510), at a first time instant 540, the pulsatility in the finger disappears due to the finger cuff pressure, which corresponds to the point 542 on the second trace 520 where oscillation stops, the pneumatic pressure 544 within the bladder 212 corresponds to the systolic arterial pressure. In this example, the pressure (e.g., the third pressure) is measured and may be used to calibrate the continuous arterial blood pressure measurement system. Similarly, when the bladder 212 is deflated (the falling segment of the first trace 510), at a second time instant 550 that the pulsatility in the finger resumes, which corresponds to the point 552 on the second trace 520 where oscillation reappears, the pneumatic pressure 554 within the bladder 212 also corresponds to the systolic arterial pressure. In this example, the pressure (e.g., the fourth pressure) is measured and may be used to calibrate the continuous arterial blood pressure measurement system. Further, in one embodiment, an average of these measured pressures (e.g., third and fourth pressures) may be used to determine the finger systolic arterial blood pressure that may be used to calibrate the continuous arterial blood pressure measurement system.

Therefore, according to the descriptions with reference to FIGS. 4 and 5, a finger systolic arterial blood pressure may be obtained by proper measurements and may be used to calibrate the continuous arterial blood pressure measurement system that operates according to the volume clamp method.

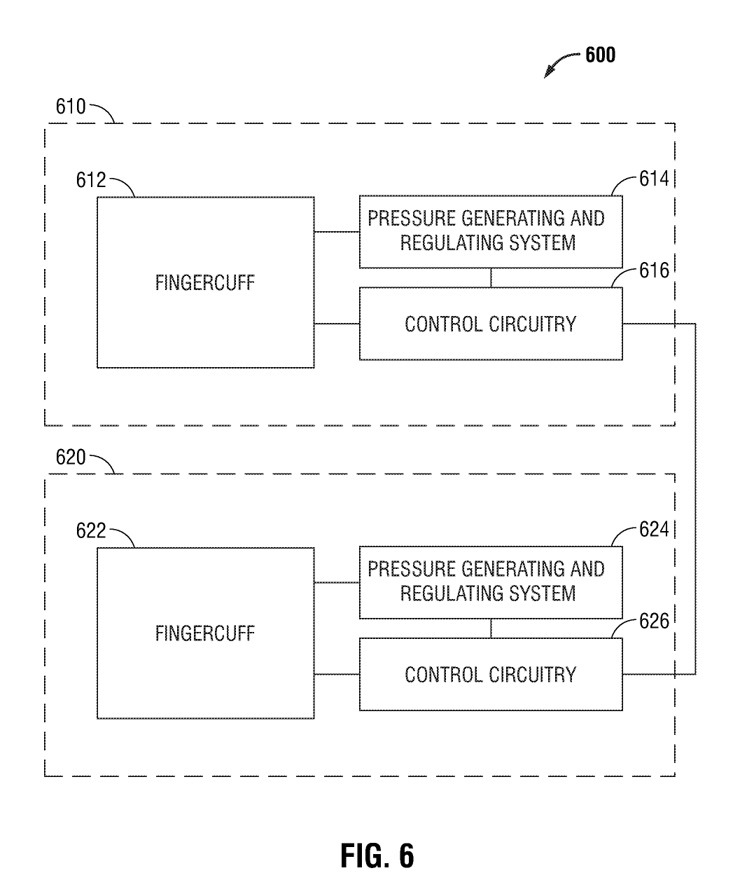

Referring to FIG. 6, a block diagram illustrating an example environment 600 in which embodiments of the invention may be practiced is shown. The environment 600 may comprise two finger arterial blood pressure measurement systems each comprising a finger cuff, a pressure generating and regulating system, and control circuitry: the first system 610 comprises a finger cuff 612, a pressure generating and regulating system 614, and control circuitry 616, and the second system 620 comprises a finger cuff 622, a pressure generating and regulating system 624, and control circuitry 626. The control circuitry 616 of the first system 610 and the control circuitry 626 of the second system 620 may be connected so that data may be exchanged between the two systems. In one embodiment, the two systems 610, 620 may make finger arterial blood pressure measurements on the same patient. Thus, the finger cuffs 612, 622 may be placed on two separate fingers of the patient. The first system 610 may make continuous arterial blood pressure measurements using the volume clamp method, while at the same time the second system 620 may make finger systolic arterial blood pressure measurements for calibration, as previously described. Thus, the measurements made by the second system 620 may be used to calibrate the first system 610 during running measurements in tandem.

In another embodiment, a single finger cuff may be used for both continuous arterial blood pressure measurement and calibration measurement. The continuous arterial blood pressure measurement may be performed with the volume clamp method, and the calibration measurement may be performed with the example method 400 previously described. The readings obtained from the calibration measurement may be utilized for calibration during continuous arterial blood pressure measurement. Therefore, the single finger cuff and the associated system including the associated pressure generating and regulating system may switch between the two modes.

It should be appreciated that because physiological changes may occur after a period of time in the finger on which continuous arterial blood pressure measurement is performed and the physiological changes may affect measurement accuracy, periodic recalibration may be required for the continuous arterial blood pressure measurement. The periodic recalibration may be performed with either the two-finger-cuff setup or the single-finger-cuff setup, as described herein. In one embodiment, where the two-finger-cuff setup is utilized, the roles (continuous measurement/calibration measurement) of the two finger cuffs and their associated systems may be switched from time to time.

It should be further appreciated that in repeating calibrations, the pressure range (e.g., the difference between the first pressure and the systolic pressure to be measured, and/or the difference between the second pressure and the systolic pressure to be measured) may be adjusted and preferably reduced based on the initial measurement and/or readings from the continuous arterial blood pressure measurement. A reduced pressure range may lead to a reduced calibration measurement time.

Therefore, embodiments of the invention provide a method to accurately obtain systolic values of the finger arterial pressure, which is beneficial, in that systolic pressure is currently the most difficult value to accurately obtain from the finger. The measurements made according to the embodiments of the invention may be used to calibrate running volume clamp-based blood pressure measurements in tandem such that running volume clamp-based blood pressure measurements are periodically or continuously updated. Thus, the calibration can be unobtrusively performed while blood pressure measurements continue on an adjacent finger. Moreover, no brachial cuff is needed for the calibration.

It should be appreciated that aspects of the invention previously described may be implemented in conjunction with the execution of instructions or code by processors, circuitry, controllers, control circuitry, etc. (e.g., processor 310 of FIG. 3). As an example, control circuity may operate under the control of a program, algorithm, code, routine, or the execution of instructions to execute methods or processes (e.g., method 400 of FIG. 4) in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, non-transitory computer readable medium, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method for measuring systolic arterial blood pressure of a patient utilizing a finger cuff including an inflatable bladder and a pulsatility sensor that detects arterial pulsatility in a finger of the patient, the method comprising:
placing the finger cuff around the finger of the patient;
inflating the bladder of the finger cuff until the finger cuff applies a first pressure to the finger, the first pressure being greater than the systolic pressure to be measured;
deflating the bladder of the finger cuff until the finger cuff applies a second pressure to the finger, the second pressure being less than the systolic pressure to be measured;
monitoring the arterial pulsatility in the finger with the pulsatility sensor; and
obtaining a measurement of the systolic arterial blood pressure based on pressure applied to the finger by the finger cuff and the arterial pulsatility in the finger.

2. The method of claim 1, wherein obtaining the measurement of the systolic arterial blood pressure further comprises measuring a third pressure applied to the finger by the finger cuff at a first time instant when the arterial pulsatility in the finger disappears when the bladder of the finger cuff is inflated as the finger systolic arterial blood pressure.

3. The method of claim 2, wherein obtaining the measurement of the systolic arterial blood pressure further comprises measuring a fourth pressure applied to the finger by the finger cuff at a second time instant when the arterial pulsatility in the finger resumes when the bladder of the finger cuff is deflated as the finger systolic arterial blood pressure.

4. The method of claim 3, wherein obtaining the measurement of the systolic arterial blood pressure further comprises measuring an average of the third pressure and the fourth pressure as the finger systolic arterial blood pressure.

5. The method of claim 1, wherein the inflatable bladder is pneumatically connected to a pressure generating and regulating system that generates, measures, and regulates pneumatic pressure.

6. The method of claim 1, wherein the pulsatility sensor comprises a plethysmograph.

7. The method of claim 6, wherein the plethysmograph comprises a light-emitting diode (LED)—photodiode pair.

8. The method of claim 1, wherein the measurement of the systolic arterial blood pressure obtained is used by the same finger cuff to calibrate a continuous arterial blood pressure measurement system.

9. The method of claim 8, wherein the continuous arterial blood pressure measurement system comprises a system that continuously measures a patient's arterial blood pressure with a volume clamp method.

10. A system for measuring systolic arterial blood pressure of a patient, comprising:
a finger cuff including an inflatable bladder; and
a pulsatility sensor that detects arterial pulsatility in a finger of the patient, wherein measuring the systolic arterial blood pressure comprises:
placing the finger cuff around the finger of the patient;
inflating the bladder of the finger cuff until the finger cuff applies a first pressure to the finger, the first pressure being greater than the systolic pressure to be measured;
deflating the bladder of the finger cuff until the finger cuff applies a second pressure to the finger, the second pressure being less than the systolic pressure to be measured;
monitoring the arterial pulsatility in the finger with the pulsatility sensor; and
obtaining a measurement of the systolic arterial blood pressure based on pressure applied to the finger by the finger cuff and the arterial pulsatility in the finger.

11. The system of claim 10, wherein obtaining the measurement of the systolic arterial blood pressure further comprises measuring a third pressure applied to the finger by the finger cuff at a first time instant when the arterial pulsatility in the finger disappears when the bladder of the finger cuff is inflated as the finger systolic arterial blood pressure.

12. The system of claim 11, wherein obtaining the measurement of the systolic arterial blood pressure further comprises measuring a fourth pressure applied to the finger by the finger cuff at a second time instant when the arterial pulsatility in the finger resumes when the bladder of the finger cuff is deflated as the finger systolic arterial blood pressure.

13. The system of claim 12, wherein obtaining the measurement of the systolic arterial blood pressure further comprises measuring an average of the third pressure and the fourth pressure as the finger systolic arterial blood pressure.

14. The system of claim 10, wherein the inflatable bladder is pneumatically connected to a pressure generating and regulating system that generates, measures, and regulates pneumatic pressure.

15. The system of claim 10, wherein the pulsatility sensor comprises a plethysmograph.

16. The system of claim 15, wherein the plethysmograph comprises a light-emitting diode (LED)—photodiode pair.

17. The system of claim 10, wherein the measurement of the systolic arterial blood pressure obtained is used by the same finger cuff to calibrate a continuous arterial blood pressure measurement system.

18. The system of claim 17, wherein the continuous arterial blood pressure measurement system comprises a system that continuously measures a patient's arterial blood pressure with a volume clamp method.

19. A non-transitory computer-readable medium comprising code which, when executed by a processor, causes the processor to perform a method for measuring systolic arterial blood pressure of a patient utilizing a finger cuff including an inflatable bladder and a pulsatility sensor that detects arterial pulsatility in a finger of the patient, the finger cuff being placed around the finger of the patient, the method comprising:
inflating the bladder of the finger cuff until the finger cuff applies a first pressure to the finger, the first pressure being greater than the systolic pressure to be measured;
deflating the bladder of the finger cuff until the finger cuff applies a second pressure to the finger, the second pressure being less than the systolic pressure to be measured;
monitoring the arterial pulsatility in the finger with the pulsatility sensor; and
obtaining a measurement of the systolic arterial blood pressure based on pressure applied to the finger by the finger cuff and the arterial pulsatility in the finger.

20. The non-transitory computer-readable medium of claim 19, wherein obtaining the measurement of the systolic arterial blood pressure further comprises measuring a third pressure applied to the finger by the finger cuff at a first time instant when the arterial pulsatility in the finger disappears when the bladder of the finger cuff is inflated as the finger systolic arterial blood pressure.

21. The non-transitory computer-readable medium of claim 20, wherein obtaining the measurement of the systolic arterial blood pressure further comprises measuring a fourth pressure applied to the finger by the finger cuff at a second time instant when the arterial pulsatility in the finger resumes when the bladder of the finger cuff is deflated as the finger systolic arterial blood pressure.

22. The non-transitory computer-readable medium of claim 21, wherein obtaining the measurement of the systolic arterial blood pressure further comprises measuring an average of the third pressure and the fourth pressure as the finger systolic arterial blood pressure.

23. The non-transitory computer-readable medium of claim 19, wherein the inflatable bladder is pneumatically connected to a pressure generating and regulating system that generates, measures, and regulates pneumatic pressure.

24. The non-transitory computer-readable medium of claim 19, wherein the pulsatility sensor comprises a plethysmograph.

25. The non-transitory computer-readable medium of claim 24, wherein the plethysmograph comprises a light-emitting diode (LED)—photodiode pair.

26. The non-transitory computer-readable medium of claim 19, wherein the measurement of the systolic arterial blood pressure obtained is used by the same finger cuff to calibrate a continuous arterial blood pressure measurement system.

27. The non-transitory computer-readable medium of claim 26, wherein the continuous arterial blood pressure measurement system comprises a system that continuously measures a patient's arterial blood pressure with a volume clamp method.
